(19) United States
(12) Plant Patent Application Publication
(10) Pub. No.: US 2002/0188984 P1
(43) Pub. Date: Dec. 12, 2002

(54) Chrysanthemum plant named ' MOZA LA 01'

(76) Inventor: Frank Moser, Alva, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/864,913

(22) Filed: May 24, 2001

(51) Int. Cl. A01H 5/00

(52) U.S. Cl. PLT/297

(57) ABSTRACT

A new and distinct Chrysanthemum plant variety characterized by a daisy type inflorescence with pink/white color ray florets, flexible plant habit, vigorous growth habit, and consistent natural season flowering habit during late September.

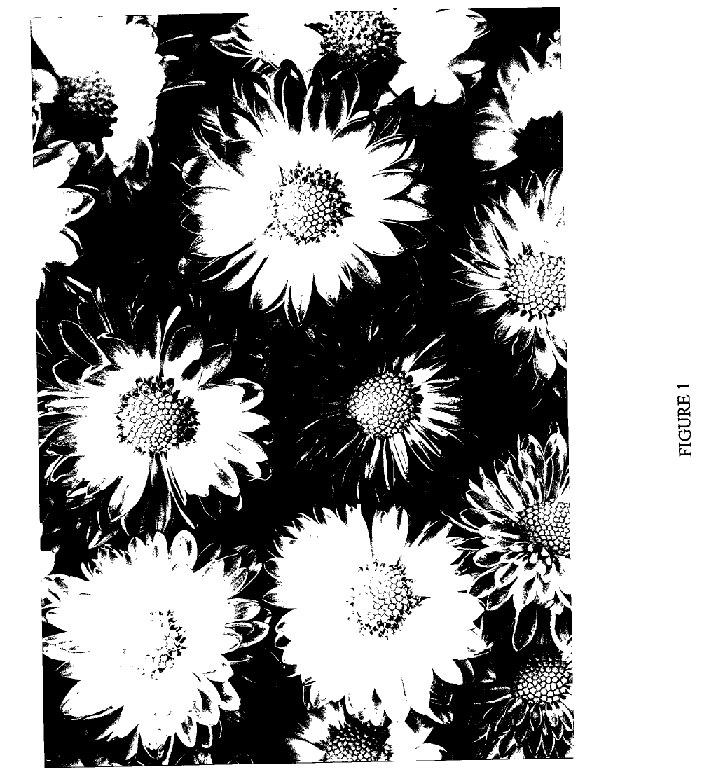

‘MOZA LA 01’: Table of R.H.S. Color Codes

| | | | |
|---|---|---|---|
| FOLIAGE | Leaf | young foliage upper surface | Near 138A |
| | | young foliage under surface | Near 138B |
| | | mature foliage upper surface | Near 137A |
| | | mature foliage under surface | Near 137B |
| | | venation upper surface | Near 138C |
| | | venation under surface | Near 137C |
| BLOOM | Ray Florets | upper surface at first opening | Near 59D |
| | | upper surface at maturity, tip | Near 70D |
| | | upper surface at maturity, base | Near 155D |
| | | upper surface at fading, tip | Near 62D |
| | | upper surface at fading, base | Near 155D |
| | | under surface at first opening | Near 54A |
| | | under surface at maturity, tip | Near 52D |
| | | under surface at maturity, base | Near 155D |
| | | under surface at fading, tip | Near 54D |
| | | under surface at fading, base | Near 155D |
| | | Pistillate: Stigma | Near 16D |
| | | Pistillate: Style | Near 16D |
| | Disc Florets | at first opening | Near 15A |
| | | at maturity | Near 15B |
| | | at fading | Near 17A |
| | | Staminate: Anther | Near 16A |
| | | Staminate: Pollen | Near 17B |
| | Peduncle | | Near 138B |
| | Inflorescence | | Near 54A |
| | Bud | | |
| | Sepals | upper surface | Near 138A |
| | | under surface | Near 138A |

### BACKGROUND OF THE INVENTION

The present invention relates to a new and distinct cultivar of Chrysanthemum plant, botanically known as Dendranthema morifolium and herein after referred to by the cultivar name ‘MOZA LA 01.’

This new cultivar originated from hybridization of a female parent Chrysanthemum cultivar (unpatented in the United States) with a male parent Chrysanthemum cultivar (unpatented in the United States). The new cultivar was discovered by the inventor in November 1998.

Asexual reproduction of the new cultivar by apical tip cutting and meristem tissue culture was performed in Oxnard, CA and has shown that the unique features of this new cultivar are stable and reproduced true to type on successive generations.

### SUMMARY OF THE INVENTION

The cultivar ‘MOZA LA 01’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘MOZA LA 01.’ These characteristics in combination distinguish ‘MOZA LA 01’ as a new and distinct Chrysanthemum:

1. Daisy type inflorescence with pink/white color ray florets;

2. Flexible plant habit;

3. Vigorous growth habit; and

4. Consistent natural season flowering habit during late September.

Plants of the new cultivar are similar to plants of the female parent cultivar in most horticultural characteristics, however plants of the new cultivar differed from plants of the female parent cultivar in the following characteristics:

1. The new cultivar has different color ray florets;

2. The new cultivar is taller; and

3. The new cultivar has a more flexible plant shape.

Plants of the new cultivar are similar to plants of the male parent cultivar in most horticultural characteristics, however plants of the new cultivar differed from plants of the male parent cultivar in the following characteristics:

1. The new cultivar has different color ray florets;

2. The new cultivar is taller; and

3. The new cultivar naturally blooms 2 weeks later.

### BRIEF DESCRIPTION OF THE PHOTOGRPAHS

The photograph shows a close up view of the flowers and buds of the new cultivar.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, CA in the month of September 2000. The growing conditions range from 60° to 65° F. at night to 70° to 85° during the day. Measurements and numerical values represent averages of typical flowering types.

Botanical classification: Dendranthema morifolium cultivar ‘MOZA LA 01’

Commercial classification: Garden-type Chrysanthemum

Parentage: Hybridization of female parent Chrysanthemum cultivar (unpatented in the United States) with a male parent Chrysanthemum cultivar (unpatented in the United States)

Propagation

Time to rooting.—7 to 14 days at approximately 21° C.

Rooting habit.—Fine, fibrous

Plant

Growth habit.—Mounding herbaceous perennial

Height.—Approximately 29 cm

Growth rate.—Moderate

Branching characteristics.—Free Branching

Length of lateral branches.—Approximately 21 cm

Number of leaves per lateral branch.—Approximately 22

Stem color.—Near 138A

Foliage

Leaf.—Arrangement: Alternate single Average Length: Approximately 7.8 cm Average Width: Approximately 4.7 cm Shape of blade: Ovate Apex: Acuminate Base: Attenuate Attachment: Stalked Margin: Serrate Texture of top surface: Lightly pubescent Texture of bottom surface: Lightly pubescent Color: young foliage upper side: Near 138A young foliage under side: Near 138B mature foliage upper side: Near 137A mature foliage under side: Near 137B venation upper side: Near 138C venation under side: Near 137C

Petiole.—Average Length: Approximately 2.9 cm

Bloom

Inflorescence.—Flowering habit: Induced by darkness period greater than 13.5 hours, approximately 49 days of appropriate day length required to induce and develop blooms Inflorescence form: Daisy Natural flowering season: Approximately the last two weeks in September Number of inflorescences per stem: Approximately 14 Inflorescence diameter: Approximately 4.5 cm Inflorescence depth: Approximately 1.5 cm Inflorescence longevity on plant. Approximately 20-24 days Persistence. Persistent

Ray florets.—Appearance: Matte Texture: Smooth Average Number perflower: Approximately 150 Shape: Oblanceolate Margin: Entire Apex: Acute Length: Approximately 2.0 cm Width: Approximately 0.8 cm Color: upper surface at first opening: Near 59D upper surface at maturity: Near 70D at the tip; Near 155D at the base upper surface at fading: Near 62D at the tip; Near 155D at the base under surface at first opening: Near 54A under surface at maturity: Near 52D at the tip; Near 55D at the base under surface at fading: Near 54D at the tip; Near 1 55D at the base

Disc florets.—Appearance: Shiny Texture: Smooth Average number perflower: Approximately 200 Shape: Cylindric Apex: Obtuse Average Length: Approximately 0.8 cm Average Width: Approximately 0.3 cm Color: at first opening: Near 15A at maturity: Near 15B atfading: Near 17A

Peduncle.—Length: at terminal end (shortest): Approximately 5 cm at lateral end (longest): Approximately 9 cm Strength: Moderate Color: Near 138B Habit: Erect

Inflorescence bud.—Length: Approximately 1 cm Diameter: Approximately 1 cm Form: Globular Color: Near 54A

Sepals.—Appearance: Matte Texture: Lightly pubescent Number: Approximately 20 to 30 Shape: Ovate Margin: Entire Apex. Acute Color: upper side: Near 138A under side: Near 138A

Reproductive Organs

Ray florets.—Pistillate: Number of pistils per flower: 1 Stigma shape: 2 branched Stigma color: Near 16D Style color: Near 16D Style length: Approximately 0.6 cm

Disc florets.—Pistillate: Number of pistils per flower: 1 Stigma shape: Cylindric Style length: Approximately 0.5 cm Staminate: Number of stamens per flower: Approximately 5 Anther shape: Tubular Anther color: Near 16A Pollen color: Near 17B

Other Characteristics

Disease resistance.—Neither resistance nor susceptibility observed

What is claimed is:

1. A new and distinct variety of chrysanthemum plant named ‘MOZA LA 01’ as herein illustrated and described.
